(19) United States
(12) Patent Application Publication
(10) Pub. No.: US 2024/0254429 A1
(43) Pub. Date: Aug. 1, 2024

(54) Simulation Apparatus, Simulation System, And Simulation Method

(71) Applicant: Terumo Kabushiki Kaisha, Tokyo (JP)

(72) Inventor: Masatsugu IGARASHI, Kanagawa (JP)

(21) Appl. No.: 18/635,158

(22) Filed: Apr. 15, 2024

Related U.S. Application Data
(63) Continuation of application No. PCT/JP2022/045323, filed on Dec. 8, 2022.

(30) Foreign Application Priority Data
Dec. 27, 2021 (JP) 2021-211858

(51) Int. Cl. C12M 1/36; C12M 1/12; C12M 1/34; G06F 30/28; G16B 5/00

(52) U.S. Cl. CPC C12M 41/48; C12M 25/10; C12M 41/30; G06F 30/28; G16B 5/00

(57) ABSTRACT

A simulation apparatus includes an acquisition unit that acquires a depletion speed at which a protein becomes depleted independently of the cells, a consumption speed at which the cells consume the protein under a first culturing condition, and condition data indicating a second culturing condition that differs from the first culturing condition; a simulation execution unit that simulates a change in a concentration of the protein accompanying propagation of the cells in response to the depletion speed, the consumption speed, and the condition data; and a display unit that acquires, as a result of the simulation, the concentration of the protein and displays whether the concentration of the protein lies within a predetermined range.

### CROSS-REFERENCE TO RELATED APPLICATIONS

The present application is a continuation of the International Patent Application No. PCT/JP2022/045323 filed Dec. 8, 2022, which designated the U.S. and claims the benefit of priority from Japanese Patent Application No. JP2021-211858 filed Dec. 27, 2021. The entire disclosures of the above-identified applications are incorporated herein by reference.

### FIELD

The present disclosure relates to a simulation apparatus, a simulation system, and a simulation method for simulating propagation of cells in a cell culturing device.

### BACKGROUND

A cell culturing device is disclosed in JP 2020-171241A. The cell culturing device is equipped with a bioreactor, a supply unit, a collection unit, and a plurality of flow paths. One portion of the flow paths forms a circulation path together with the bioreactor. The supply unit supplies a cell-containing solution and a culture medium (culturing solution) to the bioreactor. The bioreactor carries out culturing of the cells. During culturing of the cells, one portion of the culture medium (i.e., a first medium) circulates in the circulation path. During culturing of the cells, another portion of the culture medium (i.e., a second medium) is discharged as a waste liquid. The cells that have been cultured are collected by the collection unit.

In culturing of the cells, the supply of nutrients (e.g., glucose, glutamine, and/or various amino acids), the supply of gases (e.g., oxygen and/or carbon dioxide), and discharging of waste products (e.g., lactic acid and/or ammonia) is essential. Furthermore, in culturing of the cells, the supply of proteins is also essential. Therefore, bovine serum (including, for example, albumin and growth factors), growth factors, cytokines and the like are added to the culture medium.

If the proteins supplied to the cells are insufficient, the cells will not propagate. On the other hand, if a large amount of the proteins is supplied to the cells, propagation of the cells will be inhibited. Furthermore, the unit price of certain proteins (e.g., growth factors and/or cytokines) is high. For these reasons, it is desirable to appropriately control the amount of the proteins that are supplied to the cells.

### SUMMARY

A simulation apparatus configured to simulate propagation of cells in a cell culturing device is provided. The simulation apparatus may include an acquisition unit configured to acquire a depletion speed at which a predetermined protein within a culture medium becomes depleted independently of the cells and/or a consumption speed at which the cells consume the protein under a first culturing condition and/or condition data indicating a second culturing condition that differs from the first culturing condition. The simulation apparatus may include a simulation execution unit configured to simulate a change in a concentration of the protein accompanying propagation of the cells under the second culturing condition, using, for example, the depletion speed and/or the consumption speed and/or the condition data acquired by the acquisition unit. The simulation apparatus may include a display unit configured to acquire, for example, as a result of the simulation, the concentration of the protein under the second culturing condition and to display whether the concentration of the protein lies within a predetermined range.

The user of the cell culturing device may be capable of acquiring culturing conditions under which, while the amount of the protein used can be suppressed, the quality of the cells can be enhance, by designating, as a predetermined range, an optimum range for the amount of the protein to be used.

The cell culturing device may include a bioreactor, a hollow fiber membrane disposed in an interior of the bioreactor, a culturing region positioned in inner holes of the hollow fiber membrane, a non-culturing region positioned in the interior of the bioreactor and externally of the hollow fiber membranes, a first supply unit configured to supply each of a culture medium in which protein is contained and the cells to the culturing region, and a second supply unit configured to supply a basal medium in which protein is not contained to the non-culturing region. The depletion speed may include a degradation speed at which the protein degrades and an elution speed at which the protein is eluted from the culturing region into the non-culturing region.

The simulation apparatus and/or the cell culturing device may include an input unit configured to input the depletion speed and the consumption speed.

The simulation apparatus and/or the cell culturing device may include an input unit configured to input the depletion speed and a change in the concentration of the protein. The change in the concentration of the protein may be measured in the cell culturing carried out under the first culturing condition. The simulation apparatus and/or the cell culturing device may further include a calculation unit configured to calculate the consumption speed based on the concentration of the protein input by the input unit.

A simulation system configured to simulate propagation of cells in a cell culturing device is provided. The simulation system may include an acquisition unit configured to acquire a depletion speed at which a predetermined protein within a culture medium becomes depleted independently of the cells and/or a consumption speed at which the cells consume the protein under a first culturing condition and/or condition data indicating a second culturing condition that differs from the first culturing condition. The simulation system may include a simulation execution unit configured to simulate a change in a concentration of the protein accompanying propagation of the cells under the second culturing condition, using, for example, the depletion speed and/or the consumption speed and/or the condition data acquired by the acquisition unit. The simulation system may include a display unit configured to acquire, as a result of the simulation, the concentration of the protein under the second culturing condition and to display whether the concentration of the protein lies within a predetermined range.

The simulation system may further include a terminal device and a server that are capable of communicating with each other via a communication network. The terminal device may include the display unit. The server may include the acquisition unit and the simulation execution unit.

A simulation method of simulating propagation of cells in a cell culturing device is provided. The simulation method may include an acquisition step of acquiring a depletion speed at which a predetermined protein within a culture medium becomes depleted independently of the cells and/or a consumption speed at which the cells consume the protein under a first culturing condition and/or condition data indicating a second culturing condition that differs from the first culturing condition. The simulation method may include a simulation execution step of simulating a change in a concentration of the protein accompanying propagation of the cells under the second culturing condition, using, for example, the depletion speed and/or the consumption speed and/or the condition data acquired in the acquisition step. The simulation method may include a display step of acquiring, as a result of the simulation, the concentration of the protein under the second culturing condition and displaying whether the concentration of the protein lies within a predetermined range.

### DRAWINGS

FIG. 1 is a diagram illustrating the configuration of a cell culturing system in accordance with at least one example embodiment;

FIG. 2 is a diagram illustrating the configuration of a control unit of a cell culturing device in accordance with at least one example embodiment;

FIG. 3 is a diagram illustrating the configuration of a simulation apparatus in accordance with at least one example embodiment;

FIG. 4 is a diagram illustrating an input screen that is displayed on a display unit in accordance with at least one example embodiment;

FIG. 5 is a diagram illustrating an input screen that is displayed on the display unit in accordance with at least one example embodiment;

FIG. 6 is a diagram illustrating a propagation data screen that is displayed on the display unit in accordance with at least one example embodiment;

FIG. 7 is a diagram illustrating a feedback condition screen that is displayed on the display unit in accordance with at least one example embodiment;

FIG. 8 is a diagram illustrating a results screen that is displayed on the display unit in accordance with at least one example embodiment;

FIG. 9 is a diagram illustrating a results screen that is displayed on the display unit in accordance with at least one example embodiment;

FIG. 10 is a flow chart illustrating a process flow of a cell culturing method performed using the cell culturing system in accordance with at least one example embodiment;

FIG. 11 is a flow chart illustrating a process flow of cell culturing performed using the cell culturing device in accordance with at least one example embodiment;

FIG. 12 is a diagram illustrating operations of the cell culturing device at a time of cell culturing in accordance with at least one example embodiment;

FIG. 13 is a diagram illustrating operations of the cell culturing device at a time of cell stripping in accordance with at least one example embodiment;

FIG. 14 is a diagram illustrating operations of the cell culturing device at a time of cell collection in accordance with at least one example embodiment;

FIG. 15 is a diagram illustrating the configuration of another embodiment of the simulation apparatus in accordance with at least one example embodiment; and

FIG. 16 is a diagram illustrating the configuration of a simulation system in accordance with at least one example embodiment.

### DETAILED DESCRIPTION

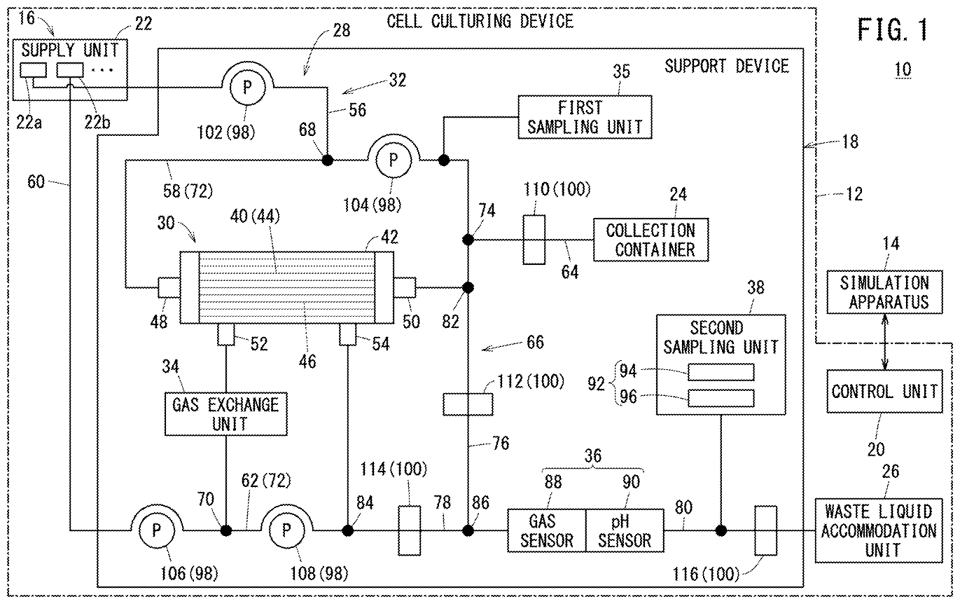

FIG. 1 is a diagram illustrating a configuration of a cell culturing system 10. The cell culturing system 10 cultures (propagates) within a culture medium cells that have been separated from living tissue. In at least one example embodiment, the cells used in the cell culturing system 10 may be adherent cells. In at least one example embodiment, the cells used in the cell culturing system 10 may be planktonic cells. In at least one example embodiment, the cells used in the cell culturing system 10 may include embryonic stem (ES) cells, induced pluripotent stem (iPS) cells, mesenchymal stem cells, and the like. The cells used in the cell culturing system 10 are not limited to the cell types described above.

The cell culturing system 10 may include a cell culturing device 12 and a simulation apparatus 14. The cell culturing device 12 may include a cell culturing circuit 16, a support device 18, and a control unit 20. A liquid may flow through the cell culturing circuit 16. Such a liquid may include at least one of a cell solution, a culture medium, a cleaning solution, and a stripping solution. The cell solution may include a solution containing cells. The culture medium may include a culturing solution for causing the cells to propagate. The culture medium may be selected depending on the cells to be cultured. The culture media may include a basal medium, a culture medium, or a combination thereof. The basal medium may include minimum essential media (MEM). In at least one example embodiment, he culture medium may include a protein-containing basal medium. The proteins may include albumin, growth factors, cytokines, and the like. For example, in at least one example embodiment, bovine serum containing albumin, growth factors, and the like may be added to the basal medium. The cleaning solution may be selected to clean the interior of the cell culturing circuit 16. As the cleaning solution, for example, water, a buffer solution, or a physiological saline solution or the like may be used. As examples of the buffer solution, there may be cited phosphate buffered saline (PBS) and tris-buffered saline (TBS) or the like. The stripping solution may be selected to strip the cells from a later-described bioreactor 30 of the cell culturing circuit 16. The stripping solution may include, for example, trypsin or an ethylenediaminetetraacetic acid (EDTA) solution. The culture medium, the cleaning solution, and the stripping solution are not limited to the liquids described above.

The cell culturing circuit 16 may be discarded after one single use thereof. For example, the cell culturing circuit 16 may be discarded each time a predetermined number of cells has been cultured. The cell culturing circuit 16 may be a disposable product. The cell culturing circuit 16 may include a supply unit 22, a collection container 24, a waste liquid accommodation unit 26, and a culturing body 28.

The supply unit 22 may supply the cell solution, the culture medium, the cleaning solution, the stripping solution, and the like to the culturing body 28. The supply unit 22 may include a first supply unit 22a and a second supply unit 22b. The collection container 24 may collect the cells that are cultured in the culturing body 28. The waste liquid accommodation unit 26 may accommodate the waste liquid that is generated in the culturing body 28. The collection container 24 and the waste liquid accommodation unit 26 may both include, for example, a medical bag. The medical bay may be obtained by molding a soft resin material into a bag-like shape. The collection container 24 and the waste liquid accommodation unit 26 may both include a tank or the like formed using a hard material.

The culturing body 28 may include a bioreactor 30, flow paths 32, a gas exchange unit 34, a first sampling unit 35, a sensor unit 36, and a second sampling unit 38.

The bioreactor 30 may include a plurality of hollow fiber membranes 40 and a cylindrical housing 42. The plurality of hollow fiber membranes 40 may be accommodated inside the housing 42. One end part (e.g., a first end part) of the respective hollow fiber membranes 40 may be fixed to one end part (e.g., a first end part) of the housing 42. Another end part (e.g., a second end part) of the respective hollow fiber membranes 40 may be fixed to another end part (e.g., a second end part) of the housing 42. The respective hollow fiber membranes 40 may include, for example, a polymer material.

The bioreactor 30 may be provided with a first region 44 and a second region 46. The first region 44 of the bioreactor 30 may be defined by inner holes of the plurality of hollow fiber membranes 40. The first region 44 may be a region of the culture medium in which the cells are present. This region may also be referred to as a culturing region. The second region 46 of the bioreactor may be defined by a space between an inner peripheral surface of the housing 42 and outer peripheral surfaces of the plurality of hollow fiber membranes 40. The second region 46 may be a non-culturing region in an interior region of the bioreactor 30. Each of the hollow fiber membranes 40 may include a plurality of non-illustrated pores therein. The first region 44 and the second region 46 may communicate with each other through the plurality of pores of the respective hollow fiber membranes 40. The pores may have an average diameter that allows low molecular-weight compounds (for example, water, ions, oxygen, lactic acid, etc.) to pass therethrough, while preventing the passage of high molecular-weight compounds (cells, etc.) therethrough. In at least one example embodiment, the pores may have an average diameter greater than or equal to 0.005 micrometers to less than or equal to 10 micrometers.

A first inlet port 48, a first outlet port 50, a second inlet port 52, and a second outlet port 54 may be installed in the housing 42. The first inlet port 48 may be installed at one end of the housing 42. The first inlet port 48 may communicate with the first region 44 via an inlet positioned at one end of the plurality of hollow fiber membranes 40. The first outlet port 50 may be installed at another end of the housing 42. The first outlet port 50 may communicate with the first region 44 via an outlet positioned at the other end of the plurality of hollow fiber membranes 40.

The second inlet port 52 and the second outlet port 54 may be installed on an outer peripheral surface of the housing 42. The second inlet port 52 may be positioned between a center of the housing 42 and the first inlet port 48 in the longitudinal direction of the housing 42. The second outlet port 54 may be positioned between the center of the housing 42 and the first outlet port 50 in the longitudinal direction of the housing 42. Each of the second inlet port 52 and the second outlet port 54 may communicate with the second region 46.

The flow paths 32 may include a plurality of tubes through which the liquids flow. The respective tubes may include a soft resin material. The flow paths 32 may include a first supply flow path 56, a first circulation flow path 58, a second supply flow path 60, a second circulation flow path 62, a collection flow path 64, and a waste liquid flow path 66.

One end of the first supply flow path 56 may be connected to the first supply unit 22a. The first supply unit 22a may supply the cell solution, the culture medium, the cleaning solution, and the stripping solution to the first supply flow path 56. In at least one example embodiment, the first supply unit 22a may supply the cell solution, the culture medium, the cleaning solution, and the stripping solution to the first supply flow path 56 one at a time at predetermined points in time. Another end of the first supply flow path 56 may be connected to a first merging section 68 within the first circulation flow path 58.

The first merging section 68 may be positioned in an intermediate portion in the direction in which the first circulation flow path 58 extends. One end of the first circulation flow path 58 may be connected to the first inlet port 48. Another end of the first circulation flow path 58 may be connected to the first outlet port 50. The first circulation flow path 58 may communicate with the inner holes (the first region 44) of the plurality of hollow fiber membranes 40.

One end of the second supply flow path 60 may be connected to the second supply unit 22b. The second supply unit 22b may supply the culture medium and the cleaning solution to the second supply flow path 60. In at least one example embodiment, the second supply unit 22b may supply the culture medium and the cleaning solution to the second supply flow path 60 one at a time at predetermined points in time. Another end of the second supply flow path 60 may be connected to a second merging section 70 within the second circulation flow path 62.

The second merging section 70 may be positioned in an intermediate portion in the direction in which the second circulation flow path 62 extends. One end of the second circulation flow path 62 may be connected to the second inlet port 52. Another end of the second circulation flow path 62 may be connected to the second outlet port 54. The second circulation flow path 62 may communicate with a space (the second region 46) between the plurality of hollow fiber membranes 40 and the housing 42. The first circulation flow path 58 and the second circulation flow path 62 may be collectively referred to as “circulation flow paths 72”.

The collection flow path 64 may extend from the first circulation flow path 58. One end of the collection flow path 64 may be connected to a collection branching section 74 within the first circulation flow path 58. The collection branching section 74 may be positioned between the first merging section 68 and the first outlet port 50 in the first circulation flow path 58. Another end of the collection flow path 64 may be connected to the collection container 24.

The waste liquid flow path 66 may enable the liquid discarded from the circulation flow paths 72 to flow therethrough. The waste liquid flow path 66 may include a first waste liquid flow path 76, a second waste liquid flow path 78, and a third waste liquid flow path 80. The first waste liquid flow path 76 may extend from the first circulation flow path 58. One end of the first waste liquid flow path 76 may be connected to a first branching section 82 within the first circulation flow path 58. The first branching section 82 may be positioned between the first outlet port 50 and the collection branching section 74 within the first circulation flow path 58. The second waste liquid flow path 78 may extend from the second circulation flow path 62. One end of the second waste liquid flow path 78 may be connected to a second branching section 84 within the second circulation flow path 62. The second branching section 84 may be positioned between the second merging section 70 and the second outlet port 54 within the second circulation flow path 62. Another end of the first waste liquid flow path 76 and another end of the second waste liquid flow path 78 may be connected together mutually at an intermediate merging section 86. One end of the third waste liquid flow path 80 may be connected at the intermediate merging section 86 to the first waste liquid flow path 76 and the second waste liquid flow path 78. Another end of the third waste liquid flow path 80 may be connected to the waste liquid accommodation unit 26.

The gas exchange unit 34 may be installed within the second circulation flow path 62 between the second merging section 70 and the second inlet port 52. The gas exchange unit 34 may allow a gas having predetermined components to pass through the liquid (the basal medium) that flows through the second circulation flow path 62. The gas used in the gas exchange unit 34 may include, for example, components therein that are similar to those of natural air. In at least one example embodiment, the gas may include nitrogen oxygen, and carbon dioxide. For example, the gas may include, for example, 75% nitrogen, 20% oxygen, and 5% carbon dioxide by volume.

The first sampling unit 35 may be connected to the first circulation flow path 58. The first sampling unit 35 may extract one portion of the liquid (the culture medium) that flows through the first circulation flow path 58. The first sampling unit 35 may collect tube fragments in which an internal liquid is contained from a sufficiently long tube using an aseptic joining device.

The sensor unit 36 may be installed in the third waste liquid flow path 80. The sensor unit 36 may include a gas sensor 88 and a pH sensor 90. The gas sensor 88 may measure a gas concentration of the liquid that flows through the third waste liquid flow path 80. For example, the gas sensor 88 may include an oxygen sensor and a carbon dioxide sensor. The oxygen sensor may measure an oxygen concentration of the liquid that flows through the third waste liquid flow path 80. The carbon dioxide sensor may measure a carbon dioxide concentration of the liquid that flows through the third waste liquid flow path 80. The pH sensor 90 may measure a pH (hydrogen ion index) of the liquid that flows through the third waste liquid flow path 80. Each of the gas sensor 88 and the pH sensor 90 may output measurement results to the control unit 20.

The second sampling unit 38 may be connected to a portion within the third waste liquid flow path 80 between the sensor unit 36 and the waste liquid accommodation unit 26. The second sampling unit 38 may be configured to extract one portion of the liquid that flows through the third waste liquid flow path 80 and to measure the components contained in the liquid. The second sampling unit 38 may include a biosensor 92, a flow path (not shown), and the like.

The biosensor 92 may include, for example, a glucose sensor 94 and a lactic acid sensor 96. The glucose sensor 94 may measure a glucose concentration of the liquid extracted from the third waste liquid flow path 80. The lactic acid sensor 96 may measure a lactic acid concentration of the liquid extracted from the third waste liquid flow path 80. Each of the glucose sensor 94 and the lactic acid sensor 96 may output measurement results to the control unit 20.

The cell culturing circuit 16 may be set in the support device 18. The support device 18 may include a cassette that supports the cell culturing circuit 16. The support device 18 may be a reusable product that is capable of being used a plurality of times.

The support device 18 may include a plurality of pumps 98 and a plurality of clamps 100. Each of the plurality of pumps 98 may impart a flowing force to the liquids inside the flow paths 32 by squeezing the wall parts of the flow paths 32. Each of the plurality of pumps 98 may include a pressing member (not shown). The pressing member may include, for example, a rotating member and a plurality of pressing rollers. The plurality of pressing rollers may be attached to an outer circumferential portion of the rotating member. The plurality of pressing rollers may be arranged at intervals with spaces left therebetween in the circumferential direction of the rotating member. Each of the pressing rollers may rub against the outer surfaces of the wall parts of the flow paths 32.

The plurality of pumps 98 may include a first supply pump 102, a first circulation pump 104, a second supply pump 106, and a second circulation pump 108. As illustrated in FIG. 1, a state in which the cell culturing circuit 16 is set in the support device 18 may be simply referred to as a “set state”.

In the set state, a portion of the first supply flow path 56 may be installed on the first supply pump 102. The first supply pump 102 may impart a flowing force to the liquid inside the first supply flow path 56 in a direction from the supply unit 22 toward the first circulation flow path 58.

In the set state, a portion of the first circulation flow path 58 may be installed on the first circulation pump 104. The first circulation pump 104 may impart a flowing force to the liquid inside the first circulation flow path 58 in a direction from the first outlet port 50 toward the first inlet port 48. Moreover, the first circulation pump 104 may impart a flowing force to the liquid inside the first circulation flow path 58 in a direction from the first inlet port 48 toward the first outlet port 50.

In the set state, a portion of the second supply flow path 60 may be installed on the second supply pump 106. The second supply pump 106 may impart a flowing force to the liquid inside the second supply flow path 60 in a direction from the supply unit 22 toward the second circulation flow path 62.

In the set state, a portion of the second circulation flow path 62 may be installed on the second circulation pump 108. The second circulation pump 108 may impart a flowing force to the liquid inside the second circulation flow path 62 in a direction from the second outlet port 54 toward the second inlet port 52. Moreover, the second circulation pump 108 may impart a flowing force to the liquid inside the second circulation flow path 62 in a direction from the second inlet port 52 toward the second outlet port 54.

The plurality of clamps 100 may close the flow paths 32 by pressing the outer surfaces toward the inner surfaces of the flow paths 32. For example, the plurality of clamps 100 may serve as on/off valves. The plurality of clamps 100 may include, for example, a collection clamp 110, a first waste liquid clamp 112, a second waste liquid clamp 114, and a third waste liquid clamp 116.

In the set state, a portion of the collection flow path 64 may be installed in the collection clamp 110. The collection clamp 110 may open and close the collection flow path 64. In the set state, a portion of the first waste liquid flow path 76 may be installed in the first waste liquid clamp 112. The first waste liquid clamp 112 may open and close the first waste liquid flow path 76. In the set state, a portion of the second waste liquid flow path 78 may be installed in the second waste liquid clamp 114. The second waste liquid clamp 114 may open and close the second waste liquid flow path 78. In the set state, a portion of the third waste liquid flow path 80 may be installed in the third waste liquid clamp 116. The third waste liquid clamp 116 may open and close the third waste liquid flow path 80.

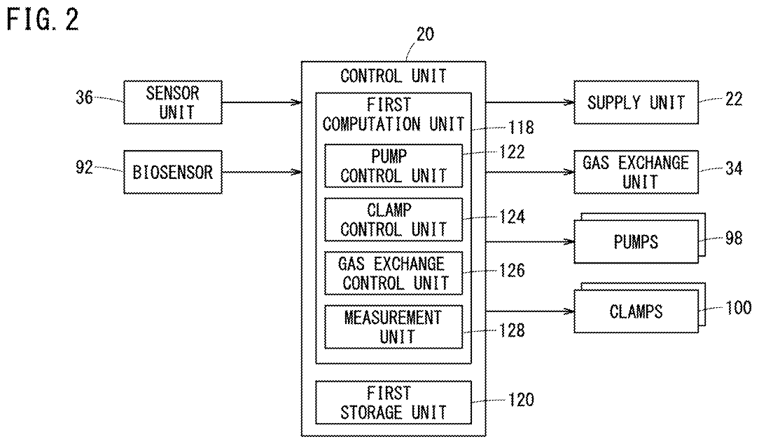

FIG. 2 is a diagram illustrating the configuration of the control unit 20 of the cell culturing device 12. The control unit 20 may include a first computation unit 118, a first storage unit 120, and various drive circuits (not shown).

The first computation unit 118 may include a processing circuit. The processing circuit may include a processor, such as a CPU or the like. The processing circuit may include an integrated circuit, such as an ASIC, an FPGA, or the like. The processor may be capable of executing various processes by executing programs stored in the first storage unit 120. The control unit 20 may function, for example, as a pump control unit 122, a clamp control unit 124, a gas exchange control unit 126, and a measurement unit 128. At least a portion from among the processes may be performed by an electronic circuit including a discrete device.

The pump control unit 122 may control each of the plurality of pumps 98. For example, the pump control unit 122 may output command signals to a pump drive circuit (not shown). The pump drive circuit may supply power to each of the plurality of pumps 98 in accordance with the command signals from the pump control unit 122. The clamp control unit 124 may control the plurality of clamps 100. Specifically, the clamp control unit 124 may output command signals to a clamp drive circuit (not shown). The clamp drive circuit may supply power to each of the plurality of clamps 100 in accordance with the command signals from the clamp control unit 124. The gas exchange control unit 126 may control the gas exchange unit 34. Specifically, the gas exchange control unit 126 may output command signals to a gas exchanger drive circuit (not shown). The gas exchanger drive circuit may supply electrical power to the gas exchange unit 34 in accordance with the command signals from the gas exchange control unit 126. The measurement unit 128 may acquire, for example, the measurement results from each of the gas sensor 88, the pH sensor 90, the glucose sensor 94, and the lactic acid sensor 96. The measurement unit 128 may cause the first storage unit 120 to store the acquired measurement results.

The first storage unit 120 may include a volatile memory and a non-volatile memory. In at least one example embodiment, the volatile memory may include, for example, a RAM or the like. The volatile memory may be used as a working memory of the processor. In the volatile memory, data and the like required for carrying out processing or computations may be temporarily stored. In at least one example embodiment, the non-volatile memory may include, for example, a ROM, a flash memory, or the like. Such a non-volatile memory may be used as a storage memory. Programs, tables, and maps, etc. may be stored in the non-volatile memory. At least a portion of the first storage unit 120 may be provided in the above-described processor, the integrated circuit, or the like.

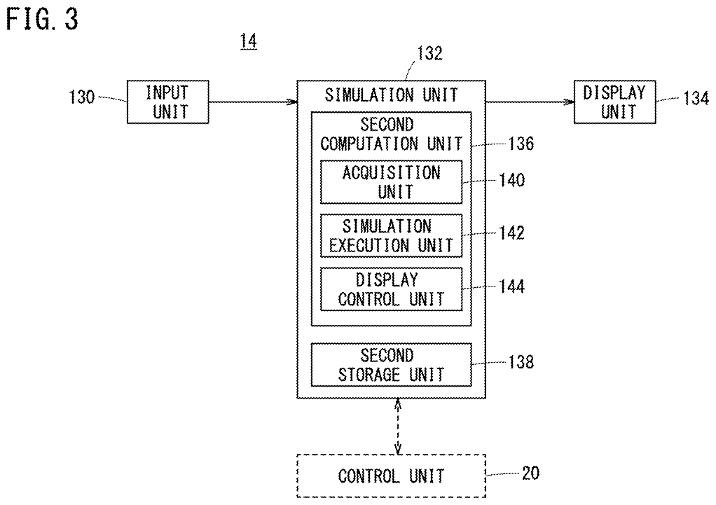

FIG. 3 is a diagram illustrating the configuration of the simulation apparatus 14. The simulation apparatus 14 may have an input unit 130, a simulation unit 132, and a display unit 134. A personal computer, a smart phone, a tablet, or the like may be used as the simulation apparatus 14.

The input unit 130 may include a human-machine interface such as a keyboard, a mouse, a touch pad, or the like. Further, the input unit 130 may include a human-machine interface that is integrated with the display unit 134, as in the form of a touch panel. The input unit 130 may be capable of inputting data to the simulation unit 132 corresponding to operations performed by the user.

The simulation unit 132 may include a second computation unit 136 and a second storage unit 138. The first computation unit 118 and the first storage unit 120 may also be used as the second computation unit 136 and the second storage unit 138. For example, the control unit 20 of the cell culturing device 12 may be used as the simulation unit 132. The second computation unit 136 may include a processing circuit. The processing circuit may be a processor such as a CPU or the like. The processing circuit may be an integrated circuit such as an ASIC, an FPGA, or the like. The processor may be capable of executing various processes by executing programs stored in the second storage unit 138. The simulation unit 132 may function as an acquisition unit 140, a simulation execution unit 142, and a display control unit 144. At least a portion from among the processes may be performed by an electronic circuit including a discrete device.

The acquisition unit 140 may acquire data from the exterior of the second computation unit 136. For example, the acquisition unit 140 may be capable of acquiring data from the input unit 130. Further, the acquisition unit 140 may be capable of acquiring data designated by the input unit 130 from the second storage unit 138. Further, the acquisition unit 140 may be capable of acquiring data designated by the input unit 130 from a device (the control unit 20 or the like) specified by the input unit 130. The simulation execution unit 142 may use the data acquired by the acquisition unit 140 to simulate the propagation of cells and changes in the protein concentration due to the cell culturing device 12. The display control unit 144 may cause the display unit 134 to display various screens. For example, the display control unit 144 can cause the display unit 134 to display the data stored in the second storage unit 138. The display control unit 144 can cause the display unit 134 to display the results of the simulation executed by the simulation execution unit 142.

The second storage unit 138 may include a volatile memory and a non-volatile memory. In at least one example embodiment, the volatile memory may include a RAM or the like. The volatile memory may be used as a working memory of the processor. In the volatile memory, data and the like required for carrying out processing or computations may be temporarily stored therein. In at least one example embodiment, the non-volatile memory may include a ROM, a flash memory, or the like. Such a non-volatile memory may be used as a storage memory. Programs, tables, and maps, etc. may be stored in the non-volatile memory. the non-volatile memory may store a simulation program that is executed by the simulation execution unit 142. Furthermore, the non-volatile memory may store default values for various data relating to cell growth. At least a portion of the second storage unit 138 may be provided in the above-described processor, the integrated circuit, or the like.

The display unit 134 may include a human-machine interface, such as a display or the like. Further, the display unit 134 may include a human-machine interface that is integrated with the input unit 130, for example, in the form of a touch panel. The display unit 134 may be capable of displaying various screens, such as described below.

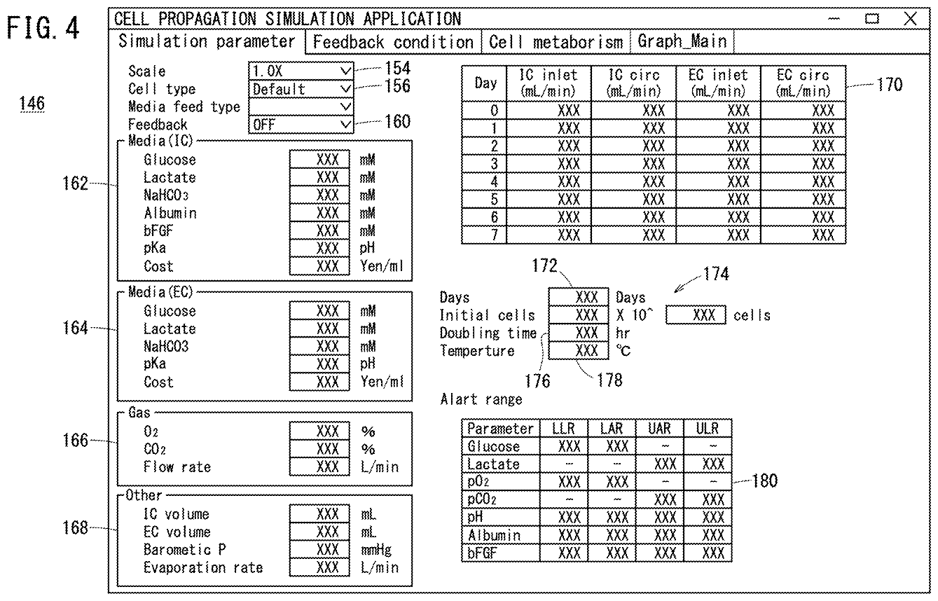

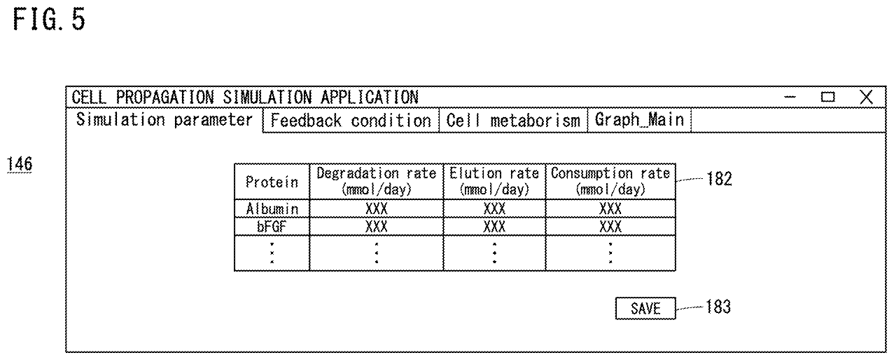

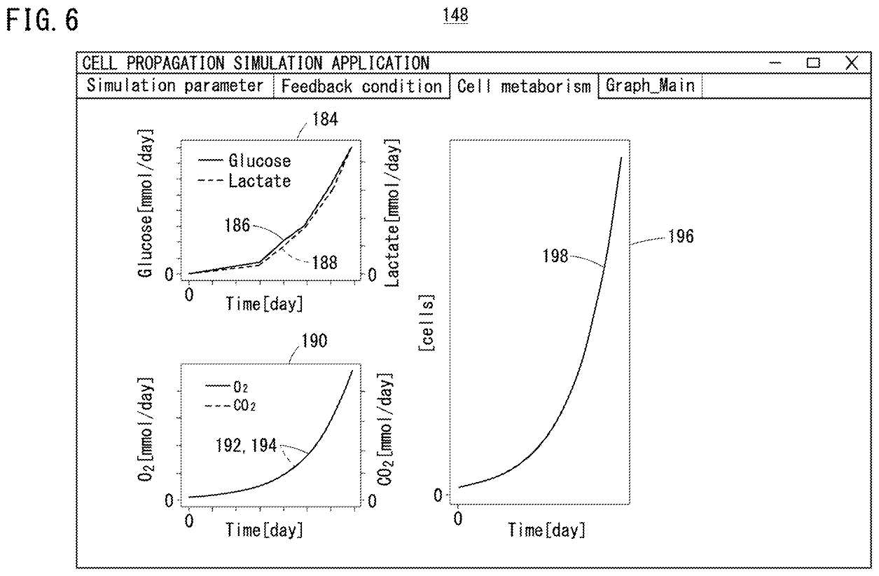

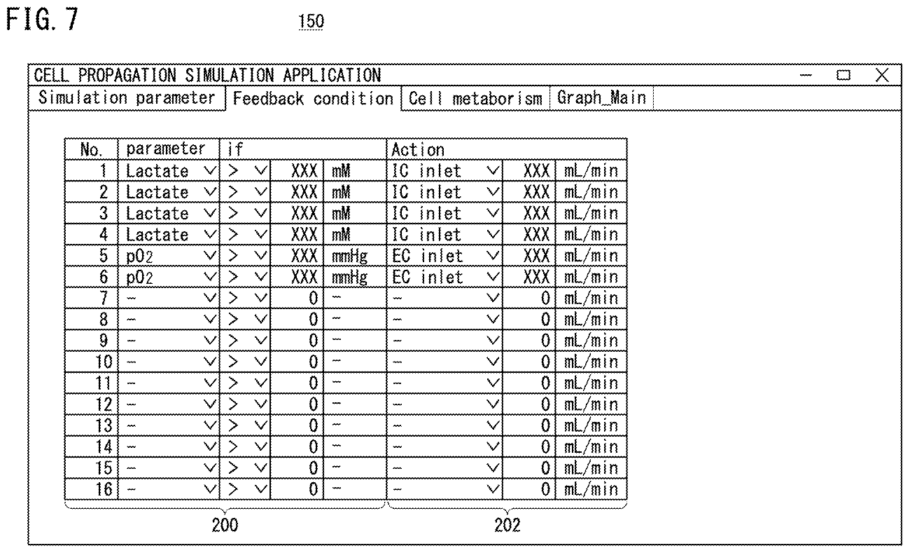

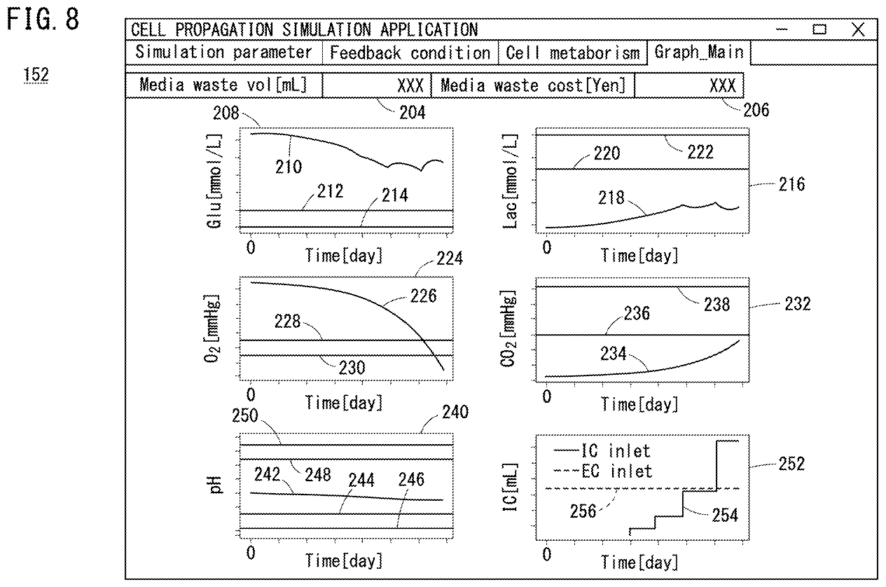

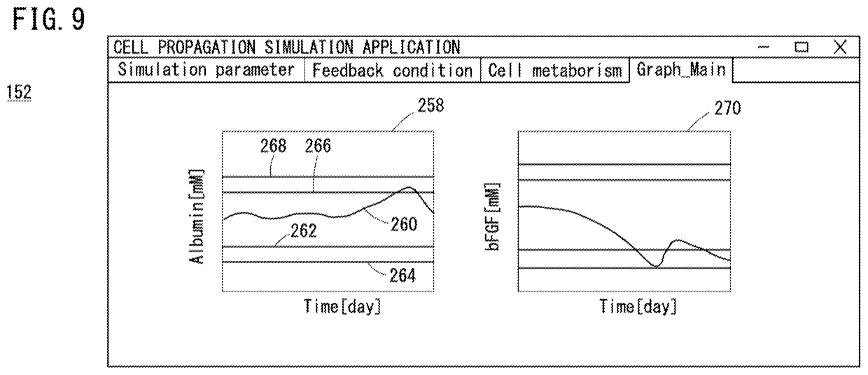

The display unit 134 may be capable of displaying an input screen 146 (see FIGS. 4 and 5), a propagation data screen 148 (see FIG. 6), a feedback condition screen 150 (see FIG. 7), a results screen 152 (see FIGS. 8 and 9), and the like.

FIG. 4 and FIG. 5 are diagrams showing the input screen 146 that is displayed on the display unit 134. FIG. 4 shows an upper portion of the input screen 146. FIG. 5 shows a lower portion of the input screen 146. In a state in which the input screen 146 of FIG. 4 is displayed on the display unit 134, when the user performs a downward scrolling operation, the input screen 146 of FIG. 5 may be displayed on the display unit 134. The input screen 146 may be a screen in order for various data used in the simulation of the cell culturing to be input. By the user operating the input unit 130, the display unit 134 displays the input screen 146.

The input screen 146 may include a scale field 154 (see FIG. 4). The scale field 154 may include input field for designating a scale of the cell culturing in the simulation. The user can select a scale from within a drop-down list displayed in the scale field 154.

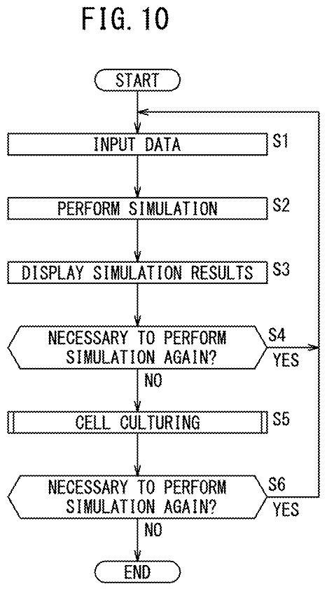

The input screen 146 may include a cell type field 156 (see FIG. 4). The cell type field 156 may be an input field for the purpose of designating propagation data that is used in the simulation. The propagation data may include data that indicate a growth state of the cells under arbitrary culturing conditions. The propagation data designated in the cell type field 156 may be a cell propagation model that is used for the simulation. The propagation data may be created on the basis of data that is actually measured in a cell culturing process carried out in the past. The second storage unit 138 may store default values of the propagation data. Further, the second storage unit 138 can store the data actually measured in the cell culturing process shown in step S5 of FIG. 10 as propagation data. A specific example of the propagation data may be shown in FIG. 6. The user can select either the default values or actually measured results from within a drop-down list displayed in the cell type field 156.

The input screen 146 may include a feedback field 160 (see FIG. 4). The feedback field 160 may include an input field for designating whether or not to use feedback conditions in the simulation. A specific example of the feedback conditions are illustrated, for example FIG. 7. The user can select either one of “ON” or “OFF” from within the drop-down list displayed in the feedback field 160. When “ON” is selected, the feedback conditions may be used in the simulation. When “OFF” is selected, the feedback conditions may not be used in the simulation.

The input screen 146 may include a culture medium input field 162 (see FIG. 4). The culture medium input field 162 may include an input field for designating data of the culture medium to be made to circulate in the first circulation flow path 58 in the simulation. The data of the culture medium may include, for example, the concentration of glucose, the concentration of lactic acid, the concentration of one or more proteins, a type of the culture medium, a pKa value, a unit price of the culture medium, and the like. One type of protein may be used or a plurality of types may be used. The user can specify proteins to be added to the culture medium in the culture medium input field 162. The culture medium data may include condition data indicating a culturing condition for the simulation.

The input screen 146 may include a basal medium input field 164 (see FIG. 4). The basal medium input field 164 may include an input field for designating data of the basal medium to be made to circulate in the second circulation flow path 62 in the simulation. The data of the culture medium may include, for example, the concentration of glucose, the concentration of lactic acid, a type of the basal medium, a pKa value, a unit price of the basal medium, and the like. The basal medium data may include condition data indicating a culturing condition for the simulation.

The input screen 146 may include a gas input field 166 (see FIG. 4). The gas input field 166 may include an input field for the purpose of designating gas data that is used by the gas exchange unit 34 in the simulation. The gas data may include, for example, a volume ratio of oxygen contained in the gas, a volume ratio of carbon dioxide contained in the gas, a flow rate of the gas, and the like. The gas data may include condition data indicating a culturing condition for the simulation.

The input screen 146 may include an additional input field 168 (see FIG. 4). The additional input field 168 may include an input field for the purpose of designating other data in relation to the culture medium. The other data may include, for example, a volume of the first circulation flow path 58, a volume of the second circulation flow path 62, an atmospheric pressure, a water vapor pressure, and the like. The other data may include condition data indicating a culturing condition for the simulation.

The input screen 146 may include a pump speed input field 170 (see FIG. 4). The pump speed input field 170 may include an input field for the purpose of designating a flow rate for each of the pumps 98 in the simulation. The flow rates of the respective pumps 98 may be set for each day of a culturing period. The flow rates of the respective pumps 98 may include data indicating a culturing condition for the simulation.

The input screen 146 may include a number of days input field 172 (see FIG. 4). The number of days input field 172 may include an input field for designating the number of days of cell culturing in the simulation. The number of days of cell culturing may include condition data indicating a culturing condition for the simulation.

The input screen 146 may include a number of seedings input field 174 (see FIG. 4). The number of seedings input field 174 may be an input field for designating the number of seedings in the simulation. The number of seedings may include condition data indicating a culturing condition for the simulation.

The input screen 146 may include a doubling time input field 176 (see FIG. 4). The doubling time input field 176 may include an input field for designating a time period (doubling time) during which the cells are doubled in the simulation. The doubling time may include condition data indicating a culturing condition for the simulation.

The input screen 146 may include a temperature input field 178 (see FIG. 4). The temperature input field 178 may include an input field for designating an environmental temperature in the simulation. The environmental temperature may include condition data indicating a culturing condition for the simulation.

The input screen 146 may include a threshold value input field 180 (see FIG. 4). The threshold value input field 180 may include an input field for the purpose of designating threshold values for the glucose concentration, the lactic acid concentration, the oxygen partial pressure, the carbon dioxide partial pressure, the pH, and the proteins. The threshold values may include, for example, at least one of a lower limit value (LLR), a lower warning value (LAR), an upper limit value (ULR), and an upper warning value (UAR). In at least one example embodiment, only the lower limit value (LLR) and the lower warning value (LAR) may be designated. In at least one example embodiment, only the upper limit value (ULR) and the upper warning value (UAR) may be designated. In at least one example embodiment, the lower limit value (LLR), the lower warning value (LAR), the upper limit value (ULR), and the upper warning value (UAR) may be designated. The user can arbitrarily specify the threshold values.

The input screen 146 may include a protein parameter field 182 (see FIG. 5). The protein parameter field 182 may include an input field for the purpose of designating, for each of respective depletion factors, and/or the speed at which the various proteins are depleted in the simulation. There are three major factors of protein depletion. The first depletion factor may include the degradation of the proteins over time. The protein parameter field 182 may include an input field for the purpose of designating a degradation speed of the proteins. The second depletion factor may include the elution of the proteins. In the interior of the bioreactor 30, the components of the culture medium in the culturing region may pass through the respective pores of the hollow fiber membranes 40 and may be eluted into the non-culturing region. Stated otherwise, a portion of the proteins contained in the culture medium may be eluted from the culturing region into the non-culturing region. The proteins that are eluted into the non-culturing region may not contribute to culturing of the cells. The protein parameter field 182 may include an input field for the purpose of designating an elution speed of the proteins. The third depletion factor may include the consumption of the proteins by the cells. The protein parameter field 182 may include an input field for the purpose of designating a consumption speed of the proteins. The degradation speed and the elution speed may be parameters that are determined regardless of the presence or absence of the cells. The elution speed may be a parameter that is determined by the cell culturing device 12. The user may be able to designate the proteins used in the cell culturing and the three depletion speeds of the proteins in the protein parameter field 182.

A save button 183 (see FIG. 5) may include a button for saving the data designated in each of the input fields. The user may be able to press the save button 183 by operating the input unit 130. When the save button 183 is pressed, the second storage unit 138 may store the data designated in each of the input fields.

FIG. 6 is a diagram showing a propagation data screen 148 that is displayed on the display unit 134. The propagation data screen 148 may include a screen showing each of various propagation data. By the user operating the input unit 130, the display unit 134 may display the propagation data screen 148. The second storage unit 138 may store each of the propagation data as a data set.

The propagation data screen 148 may include a biodata graph 184. In the biodata graph 184, the horizontal axis may represent time and the vertical axis may represent a metabolic rate of the biodata. In the biodata graph 184, a metabolic rate line 186 and a metabolic rate line 188 may be displayed. The metabolic rate line 186 may indicate a transitioning of the metabolic rate of glucose. The metabolic rate line 188 may indicate a transitioning of the metabolic rate of lactic acid. The metabolic rates of the biodata may include propagation data that indicate the growth state of the cells.

The propagation data screen 148 may include a gas data graph 190. In the gas data graph 190, the horizontal axis may represent time and the vertical axis may represent a metabolic rate of the biodata. In the gas data graph 190, a metabolic rate line 192 and a metabolic rate line 194 may be displayed. The metabolic rate line 192 may indicate a transitioning of the metabolic rate of oxygen. The metabolic rate line 194 may indicate a transitioning of the metabolic rate of carbon dioxide. The metabolic rates of the gas data may include propagation data that indicate the growth state of the cells.

The propagation data screen 148 may include a cell graph 196. In the cell graph 196, the horizontal axis may represent time and the vertical axis may represent the number of cells. A number of cells line 198 may be displayed in the cell graph 196. The number of cells line 198 may indicate a transitioning of the number of cells. The number of cells may include propagation data that indicates the growth state of the cells.

FIG. 7 is a diagram illustrating a feedback condition screen 150 that may be displayed on the display unit 134. The feedback condition screen 150 may include a screen in order to input feedback conditions and change data. The feedback conditions may include conditions for the purpose of changing the condition data in accordance with the situation of the simulation during the simulation. The change data may include modified values of the condition data. By the user operating the input unit 130, the display unit 134 may display the feedback condition screen 150.

The feedback condition screen 150 may include a condition field 200 and a data field 202. The condition field 200 may include an input field for the purpose of designating the feedback conditions. The data field 202 may include an input field for the purpose of designating the change data. For example, the condition field 200 and the data field 202 indicated by No. 1 in FIG. 7 may imply the condition of “In the case that the lactic acid has become greater than XXX [mM], the flow rate of the first circulation pump 104 is set to XXX [mL/min]”. The feedback conditions and the change data may include condition data indicating a culturing condition for the simulation. Moreover, although not illustrated, feedback conditions, such as glucose, carbon dioxide, pH, and various proteins, etc., can also be designated.

When the save button 183 of the input screen 146 is pressed, the second storage unit 138 may store the data designated in each of the input fields of the feedback condition screen 150.

FIG. 8 and FIG. 9 are diagrams illustrating the results screen 152 that is displayed on the display unit 134. FIG. 8 illustrates an upper portion of the results screen 152, and FIG. 9 illustrates a lower portion of the results screen 152. In a state in which the results screen 152 of FIG. 8 is displayed on the display unit 134, when the user performs a downward scrolling operation, the results screen 152 of FIG. 9 may be displayed on the display unit 134. The results screen 152 may include a screen showing the results of the simulation performed in step S2 of FIG. 10. After the simulation, by the user operating the input unit 130, the display unit 134 may display the results screen 152.

The results screen 152 may include a waste amount field 204 and a cost field 206. The waste amount field 204 may display the total amount of waste of the culture medium in the cell culturing that is simulated. The cost field 206 may display the cost in the cell culturing that is simulated.

The results screen 152 may include a glucose graph 208 (see FIG. 8). In the glucose graph 208, the horizontal axis may represent time and the vertical axis may represent the glucose concentration. In the glucose graph 208, a concentration line 210, a warning line 212, and a lower limit line 214 may be displayed. The concentration line 210 may indicate a transitioning of the glucose concentration during the culturing period. The warning line 212 may indicate a boundary value between an OK range and a warning range. The lower limit line 214 may indicate a boundary value between the warning range and an NG range. The boundary value indicated by the warning line 212 may be the lower warning value of the glucose concentration that was input in the threshold value input field 180 of the input screen 146. The boundary value indicated by the lower limit line 214 may be the lower limit value of the glucose concentration that was input in the threshold value input field 180 of the input screen 146. The range above the warning line 212 may be the OK range. The range below the lower limit line 214 may be the NG range. The range between the warning line 212 and the lower limit line 214 may be the warning range. The concentration line 210 may be within the OK range above the warning line 212. At all times during the culturing period, the glucose concentration may be within the OK range.

The results screen 152 may include a lactic acid graph 216 (see FIG. 8). In the lactic acid graph 216, the horizontal axis may represent time and the vertical axis may represent the lactic acid concentration. In the lactic acid graph 216, a concentration line 218, a warning line 220, and an upper limit line 222 may be displayed. The concentration line 218 may indicate a transitioning of the lactic acid concentration during the culturing period. The warning line 220 may indicate a boundary value between an OK range and a warning range. The upper limit line 222 may indicate a boundary value between the warning range and an NG range. The boundary value indicated by the warning line 220 may be the upper warning value of the lactic acid concentration that was input in the threshold value input field 180 of the input screen 146. The boundary value indicated by the upper limit line 222 may be the upper limit value of the lactic acid concentration that was input in the threshold value input field 180 of the input screen 146. The range below the warning line 220 may be the OK range. The range above the upper limit line 222 may be the NG range. The range between the warning line 220 and the upper limit line 222 may be the warning range. The concentration line 218 preferably lies within the OK range below the warning line 220. More specifically, at all times during the culturing period, the lactic acid concentration preferably lies within the OK range.

The results screen 152 may include an O2 graph 224 (see FIG. 8). In the O2 graph 224, the horizontal axis may represent time and the vertical axis may represent the oxygen partial pressure. In the O2 graph 224, a partial pressure line 226, a warning line 228, and a lower limit line 230 may be displayed. The partial pressure line 226 may indicate a transitioning of the oxygen partial pressure during the culturing period. The warning line 228 may indicate a boundary value between an OK range and a warning range. The lower limit line 230 may indicate a boundary value between the warning range and an NG range. The boundary value indicated by the warning line 228 may be the lower warning value of the oxygen partial pressure that was input in the threshold value input field 180 of the input screen 146. The boundary value indicated by the lower limit line 230 may be the lower limit value of the oxygen partial pressure that was input in the threshold value input field 180 of the input screen 146. The range above the warning line 228 may be the OK range. The range below the lower limit line 230 may be the NG range. The range between the warning line 228 and the lower limit line 230 may be the warning range. The partial pressure line 226 may be within the OK range above the warning line 228. At all times during the culturing period, the oxygen partial pressure may be within the OK range.

The results screen 152 may include a CO2 graph 232 (see FIG. 8). In the CO2 graph 232, the horizontal axis may represent time and the vertical axis may represent the carbon dioxide partial pressure. In the CO2 graph 232, a partial pressure line 234, a warning line 236, and an upper limit line 238 may be displayed. The partial pressure line 234 may indicate a transitioning of the carbon dioxide partial pressure during the culturing period. The warning line 236 may indicate a boundary value between an OK range and a warning range. The upper limit line 238 may indicate a boundary value between the warning range and an NG range. The boundary value indicated by the warning line 236 may be an upper warning value of the carbon dioxide partial pressure that was input in the threshold value input field 180 of the input screen 146. The boundary value indicated by the upper limit line 238 may be an upper limit value of the carbon dioxide partial pressure that was input in the threshold value input field 180 of the input screen 146. The range below the warning line 236 may be the OK range. The range above the upper limit line 238 may be the NG range. The range between the warning line 236 and the upper limit line 238 may be the warning range. The partial pressure line 234 may be within the OK range below the warning line 236. At all times during the culturing period, the carbon dioxide partial pressure may be within the OK range.

The results screen 152 may include a pH graph 240 (see FIG. 8). In the pH graph 240, the horizontal axis may represent time and the vertical axis may represent the pH of the culture medium. In the pH graph 240, a pH line 242, a lower warning line 244, a lower limit line 246, an upper warning line 248, and an upper limit line 250 may be displayed. The pH line 242 may indicate a transitioning of the pH during the culturing period. The lower warning line 244 may indicate a boundary value between an OK range and a lower warning range. The lower limit line 246 may indicate a boundary value between the lower warning range and a first NG range. The upper warning line 248 may indicate a boundary value between an OK range and an upper warning range. The upper limit line 250 may indicate a boundary value between the upper warning range and a second NG range. The boundary value indicated by the lower warning line 244 may be the lower warning value of the pH that was input in the threshold value input field 180 of the input screen 146. The boundary value indicated by the lower limit line 246 may be the lower limit value of the pH that was input in the threshold value input field 180 of the input screen 146. The boundary value indicated by the upper warning line 248 may be the upper warning value of the pH that was input in the threshold value input field 180 of the input screen 146. The boundary value indicated by the upper limit line 250 may be the upper limit value of the pH that was input in the threshold value input field 180 of the input screen 146. The range between the lower warning line 244 and the upper warning line 248 may be the OK range. The range below the lower limit line 246 may be the first NG range. The range between the lower warning line 244 and the lower limit line 246 may be the lower warning range. The range above the upper limit line 250 may be the second NG range. The range between the upper warning line 248 and the upper limit line 250 may be the upper warning range. The pH line 242 may be within the OK range between the lower warning line 244 and the upper warning line 248. At all times during the culturing period, the pH of the culture medium preferably may be within the OK range.

The results screen 152 may include a flow rate graph 252 (see FIG. 8). In the flow rate graph 252, the horizontal axis may represent time and the vertical axis may represent the flow rate in the first circulation pump 104 and the flow rate in the second circulation pump 108. In the flow rate graph 252, a first flow rate line 254, and a second flow rate line 256 may be displayed. The first flow rate line 254 may indicate a transitioning of the flow rate in the first circulation pump 104 during the culturing period. The second flow rate line 256 may indicate a transitioning of the flow rate in the second circulation pump 108 during the culturing period.

The results screen 152 may include an albumin graph 258 (see FIG. 9). In the albumin graph 258, the horizontal axis may represent time and the vertical axis may represent the albumin concentration. In the albumin graph 258, a concentration line 260, a lower warning line 262, a lower limit line 264, an upper warning line 266, and an upper limit line 268 may be displayed. The concentration line 260 may indicate a transitioning of the albumin concentration during the culturing period. The lower warning line 262 may indicate a boundary value between an OK range and a lower warning range. The lower limit line 264 may indicate a boundary value between the lower warning range and the first NG range. The upper warning line 266 may indicate a boundary value between an OK range and an upper warning range. The upper limit line 268 may indicate a boundary value between the upper warning range and the second NG range. The boundary value indicated by the lower warning line 262 may be the lower warning value of the albumin concentration that was input in the threshold value input field 180 of the input screen 146. The boundary value indicated by the lower limit line 264 may be the lower limit value of the albumin concentration that was input in the threshold value input field 180 of the input screen 146. The boundary value indicated by the upper warning line 266 may be the upper warning value of the albumin concentration that was input in the threshold value input field 180 of the input screen 146. The boundary value indicated by the upper limit line 268 may be the upper limit value of the albumin concentration that was input in the threshold value input field 180 of the input screen 146. The range between the lower warning line 262 and the upper warning line 266 may be the OK range. The range below the lower limit line 264 may be the first NG range. The range between the lower warning line 262 and the lower limit line 264 may be the lower warning range. The range above the upper limit line 268 may be the second NG range. The range between the upper warning line 266 and the upper limit line 268 may be the upper warning range. The concentration line 260 may be within the OK range between the lower warning line 262 and the upper warning line 266. At all times during the culturing period, the albumin concentration may be within the OK range.

The results screen 152 may include a bFGF graph 270 (see FIG. 9). The bFGF graph 270 may indicate a transitioning of the bFGF concentration during the culturing period. Concerning the bFGF graph 270, a description thereof can be given by replacing “albumin” in the description of the albumin graph 258 described above with “bFGF”.

Moreover, it should be noted that the results screen 152 can also indicate a transitioning in the concentrations of other proteins. For example, the results screen 152 can indicate the transitioning in the concentrations of the proteins designated in the culture medium input field 162 of the input screen 146.

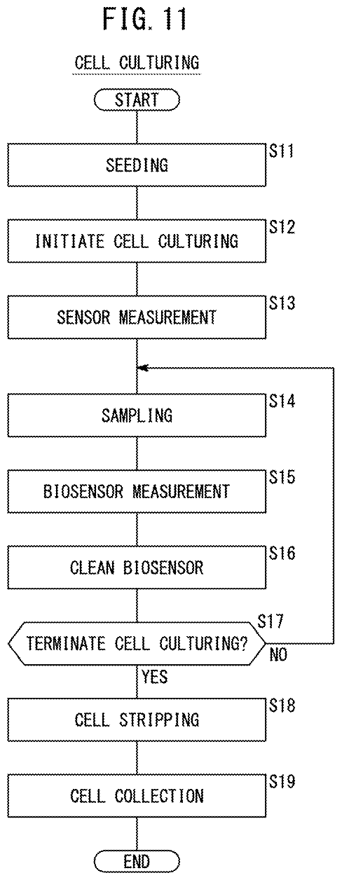

FIG. 10 is a flow chart illustrating a process flow of a cell culturing method performed using the cell culturing system 10. Step S1 to Step S3 in FIG. 10 may be performed by the simulation apparatus 14. Step S5 in FIG. 10 may be performed by the cell culturing device 12. The details of step S5 are illustrated in FIG. 11. Step S4 and step S6 may be determined by the user.

Prior to performing the simulation, the user may measure the three depletion speeds of the aforementioned proteins. For example, by an arbitrary method, the user may measure the degradation speed of the proteins. Further, in a state in which the cells are not supplied to the inner holes of the hollow fiber membranes 40, the user may measure the elution speed of the proteins. In this case, the user may use the cell culturing device 12 or may use another device equipped with the hollow fiber membranes 40.

Further, in order to measure the speed at which the proteins are consumed by the cells, the user may actually carry out culturing of the cells using the cell culturing device 12. The user may measure the concentration of the proteins within the culture medium that is extracted by the first sampling unit 35. Such a measurement may be performed, for example, using a BCA method, a Bradford method, or the like. By performing the measurement over time, the user may be able to acquire the transitioning in the concentrations of the proteins. The user can calculate the speed at which the proteins are consumed by the cells from the transitioning in the concentration of the proteins, the degradation speed of the proteins, and the elution speed of the proteins. The process from measuring the concentration of the proteins in the sampled culture medium to the calculation of the consumption speed of the proteins can also be automated.

Prior to step S1, the user may operate the input unit 130 and thereby initiate the simulation program. Responsive to the operation of the user, the second computation unit 136 may execute the simulation program that is stored in the second storage unit 138. Upon doing so, the display unit 134 may display the input screen 146 illustrated in FIG. 4 and FIG. 5.

In step S1, the user may operate the input unit 130 and thereby specify the data in each of the input fields of the input screen 146. For example, in the initial simulation, the user may specify the default values in the cell type field 156. Further, the user may input the depletion speed (the degradation speed, the elution speed, the consumption speed) of each of the proteins in the protein parameter field 182. The user may press the save button 183 after designating each item of data. The input unit 130 may input the data in each of the input fields to the simulation unit 132. The second storage unit 138 may store each of such data. After step S1 is completed, the process may proceed to step S2.

In step S2, the user may operate the input unit 130 and thereby initiate the cell culturing simulation. In response to an instruction from the input unit 130, the simulation execution unit 142 may initiate the cell culturing simulation using each item of data (the propagation data, the condition data, and the various parameters of the proteins) stored in the second storage unit 138. Using the propagation data, the condition data, and the various parameters of the proteins, the simulation execution unit 142 may simulate cell culturing for a specified culturing period. At each of respective times during the culturing period, the simulation execution unit 142 may calculate the amount of each of the components contained within the culture medium. More specifically, the simulation execution unit 142 may calculate the concentration of the glucose at each of respective times during the culturing period. Further, the simulation execution unit 142 may calculate the concentration of the lactic acid at each of respective times during the culturing period. Further, the simulation execution unit 142 may calculate the oxygen partial pressure at each of respective times during the culturing period. Further, the simulation execution unit 142 may calculate the carbon dioxide partial pressure at each of respective times during the culturing period. Further, the simulation execution unit 142 mat calculate the pH at each of respective times during the culturing period. Further, the simulation execution unit 142 may calculate the concentration of each of the proteins at each of respective times during the culturing period. The simulation execution unit 142 can calculate the amount of each of the components by way of a known calculation method. An example calculation method is described, for example, in the document, “Journal of Chemical Technology and Metallurgy, Vol. 48, Iss. 4, 2013, pp. 351-356, Experimental Determination of the Volumetric Mass Transfer Coefficient”, the entire disclosure of which is hereby incorporated by reference. The second storage unit 138 may store the calculation results of the simulation execution unit 142.

The simulation execution unit 142 may determine whether or not the feedback conditions are satisfied on the basis of each of the calculated values at each of the respective times. In the case that the feedback conditions are satisfied, the simulation execution unit 142 may change a portion of the culturing conditions in accordance with the settings of the feedback conditions. For example, the simulation execution unit 142 may change the flow rate data of any of the pumps 98. The simulation execution unit 142 may continue with the simulation using the changed data. The second storage unit 138 may store the changed condition data.

After the cell culturing simulation is completed, the simulation execution unit 142 may calculate the total amount of the culture medium consumed and the total amount of the waste of the culture medium in the simulation. Further, the simulation execution unit 142 may calculate the cost using the total amount of the culture medium consumed and the unit price of the culture medium. The second storage unit 138 may store the calculation results of the simulation execution unit 142. After step S2 is completed, the process may proceed to step S3.

In step S3, the user may operate the input unit 130 and thereby display the results of the simulation. In response to an instruction from the input unit 130, the display control unit 144 may cause the display unit 134 to display the results of the simulation. The display unit 134 may display the results screen 152 shown in FIG. 8 and FIG. 9. After step S3 is completed, the process may proceed to step S4.

In step S4, the user may determine whether or not it is necessary to perform the simulation again. In any of the graphs on the results screen 152, in the case of there being a portion that lies outside of the OK range in the transitioning of the calculated values, the user may modify the condition data and may execute the simulation again. In the case that it is necessary to perform the simulation again (step S4: YES), the process may return to step S1. On the other hand, in the case that it is not necessary to perform the simulation again (step S4: NO), the process may proceed to step S5.

In step S5, using the cell culturing device 12, the user may carry out culturing of the cells. The user may operate the input device (not shown) of the cell culturing device 12 and thereby set the culturing conditions specified in step S1 of FIG. 10. For example, in the case that the control unit 20 of the cell culturing device 12 and the simulation unit 132 of the simulation apparatus 14 may be connected by a signal line, the control unit 20 may acquire the condition data of the culturing conditions from the second storage unit 138 of the simulation unit 132. Moreover, after the cell culturing process is completed, the simulation unit 132 may acquiree the condition data of the culturing conditions and new cell propagation data from the control unit 20. The second storage unit 138 may store each of the data acquired from the control unit 20. After step S5 is completed, the process may proceed to step S6.

In step S6, the user may determine whether or not it is necessary to perform the simulation again. Culturing of the cells may be performed a plurality of times. As the number of times that cell culturing is performed increases, the user may gradually increase the scale of the cell culturing. The user may preferably perform the simulation each time that the scale of the cell culturing is made to increase. In the case that it is necessary to perform the simulation again (step S6: YES), the process may return to step S1. On the other hand, in the case that it is not necessary to perform the simulation again (step S6: NO), the culturing of the cells may be brought to an end.

FIG. 11 is a flow chart illustrating a process flow of the cell culturing performed using the cell culturing device 12. The series of steps illustrated in FIG. 11 may be carried out in step S5 illustrated in FIG. 10.

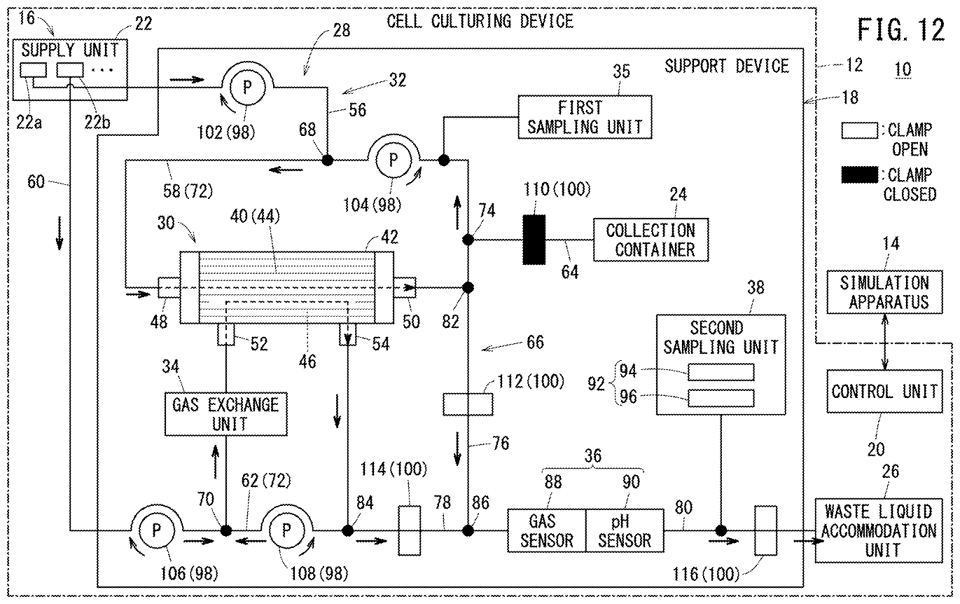

In step S11, the control unit 20 may carry out seeding. As illustrated in FIG. 12, the pump control unit 122 may control each of the pumps 98. Further, as illustrated in FIG. 12, the clamp control unit 124 may control each of the clamps 100. The control unit 20 may control the first supply unit 22a and thereby supply the cell solution to the first supply flow path 56. Upon doing so, the cell solution may be introduced from the first supply unit 22a into the first merging section 68 of the first circulation flow path 58 via the first supply flow path 56. The cell solution having been introduced into the first merging section 68 may flow from the first inlet port 48 through the first region 44 and may be guided to the first outlet port 50. In the first region 44, the cells within the cell solution may adhere to the inner surfaces of each of the hollow fiber membranes 40 of the bioreactor 30.

In step S12, the control unit 20 may initiate culturing of the cells. Specifically, the control unit 20 may control the first supply unit 22a and thereby supply the culture medium to the first supply flow path 56. Upon doing so, the culture medium may be introduced from the first supply unit 22a into the first merging section 68 of the first circulation flow path 58 via the first supply flow path 56. The culture medium having been introduced into the first merging section 68 may circulate in an annular flow path including the first circulation flow path 58, the first inlet port 48, the first region 44, and the first outlet port 50.

Further, the control unit 20 may control the second supply unit 22b and thereby supply the basal medium to the second supply flow path 60. Upon doing so, the basal medium may be introduced from the second supply unit 22b into the second merging section 70 of the second circulation flow path 62 via the second supply flow path 60. The basal medium having been introduced into the second merging section 70 may circulate in an annular flow path including the second circulation flow path 62, the second inlet port 52, the second region 46, and the second outlet port 54.

Furthermore, the gas exchange control unit 126 may control the gas exchange unit 34 and thereby carry out gas exchange on the basal medium that flows through the second circulation flow path 62. Specifically, in the gas exchange unit 34, a gas of predetermined components may pass through the basal medium prior to the basal medium flowing into the second inlet port 52. In accordance therewith, the gas concentration (the oxygen gas concentration and the carbon dioxide gas concentration) and the pH of the basal medium introduced into the second inlet port 52 of the bioreactor 30 can be adjusted to values suitable for cell culturing. In the bioreactor 30, the culture medium in the first region 44 and the basal medium in the second region 46 may be exchanged through the pores of each of the hollow fiber membranes 40. In accordance therewith, the gas concentration and the pH of the culture medium in the first region 44 may be adjusted.

Further, at an appropriate timing, the clamp control unit 124 may control the first waste liquid clamp 112 and thereby cause the first waste liquid flow path 76 to open or close. When the first waste liquid flow path 76 is opened, a portion of the culture medium inside the first circulation flow path 58 may be guided to the third waste liquid flow path 80 via the first waste liquid flow path 76. Further, at an appropriate timing, the clamp control unit 124 may control the second waste liquid clamp 114 and thereby cause the second waste liquid flow path 78 to open or close. When the second waste liquid flow path 78 is opened, a portion of the basal medium inside the second circulation flow path 62 may be guided to the third waste liquid flow path 80 via the second waste liquid flow path 78.

In step S13, the gas sensor 88 may measure the oxygen concentration of the culture medium (the culture medium+the basal medium) and the carbon dioxide concentration of the culture medium. The pH sensor 90 may measure the pH of the culture medium. The gas sensor 88 and the pH sensor 90 may transmit their measurement results to the control unit 20. The measurement unit 128 may acquire the measurement results from each of the sensors. The measurement unit 128 may cause the first storage unit 120 to store the acquired measurement results. The gas sensor 88 and the pH sensor 90 may perform measurements until the culturing of the cells is completed.

In step S14, the control unit 20 may sample the culture medium. The pump control unit 122 and the gas exchange control unit 126 may control a pump (not shown) of the second sampling unit 38 and a clamp (not shown) of the second sampling unit 38 and thereby sample the culture medium in the third waste liquid flow path 80. The sampled culture medium may pass through the biosensor 92 and flow to the waste liquid accommodation unit 26. In this instance, the first sampling unit 35 may sample the basal medium.

In step S15, the glucose sensor 94 may measure the glucose concentration of the culture medium. The lactic acid sensor 96 may measure the lactic acid in the culture medium. The glucose sensor 94 and the lactic acid sensor 96 may transmit their measurement results to the control unit 20. The measurement unit 128 may acquire the measurement results from each of the sensors. The measurement unit 128 may cause the first storage unit 120 to store the acquired measurement results. In this instance, the concentration of the basal medium subjected to sampling by the first sampling unit 35 may also be measured.

In step S16, the control unit 20 may clean the biosensor 92. One or more pumps (not shown), one or more clamps (not shown), a cleaning solution supply unit (not shown), and the like may be provided in the second sampling unit 38. The pump control unit 122 may control the pump(s) of the second sampling unit 38. Further, the clamp control unit 124 may control the clamp(s) of the second sampling unit 38. Further, the control unit 20 may control the cleaning solution supply unit. Upon doing so, the cleaning solution may flow from the cleaning solution supply unit into the biosensor 92. Consequently, the biosensor 92 may be cleaned. The cleaning solution used to clean the biosensor 92 may flow into the waste liquid accommodation unit 26.

In step S17, the control unit 20 may determine whether or not to terminate culturing of the cells based on the measurement results that were measured by the biosensor 92. In the case that the control unit 20 determines to terminate culturing of the cells (step S17: YES), the process may proceed to step S18. On the other hand, in the case that the control unit 20 determines to continue culturing of the cells (step S17: NO), the process may proceed to step S14.

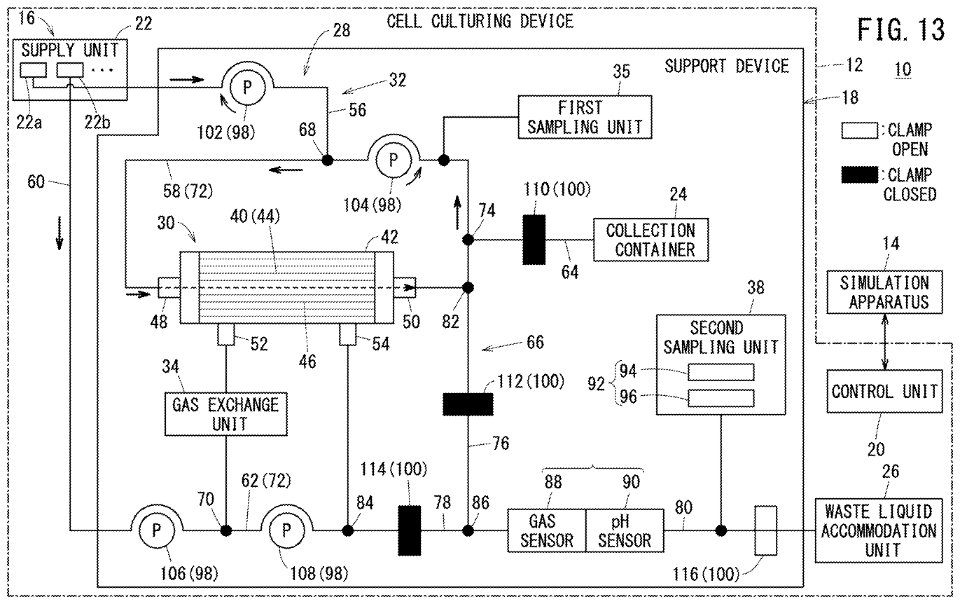

In step S18, the control unit 20 may carry out cell stripping. As illustrated in FIG. 13, the pump control unit 122 may turn off the second supply pump 106 and the second circulation pump 108. Further, as illustrated in FIG. 13, the clamp control unit 124 may control the first waste liquid clamp 112 and the second waste liquid clamp 114 and thereby close the first waste liquid flow path 76 and the second waste liquid flow path 78. Further, the control unit 20 may control the supply unit 22 and thereby supply the stripping solution to the first supply flow path 56. Upon doing so, the stripping solution may be guided from the supply unit 22 to the bioreactor 30 via the first supply flow path 56 and the first circulation flow path 58. In the bioreactor 30, the stripping solution may strip the cultured cells from the inner surfaces of each of the hollow fiber membranes 40.

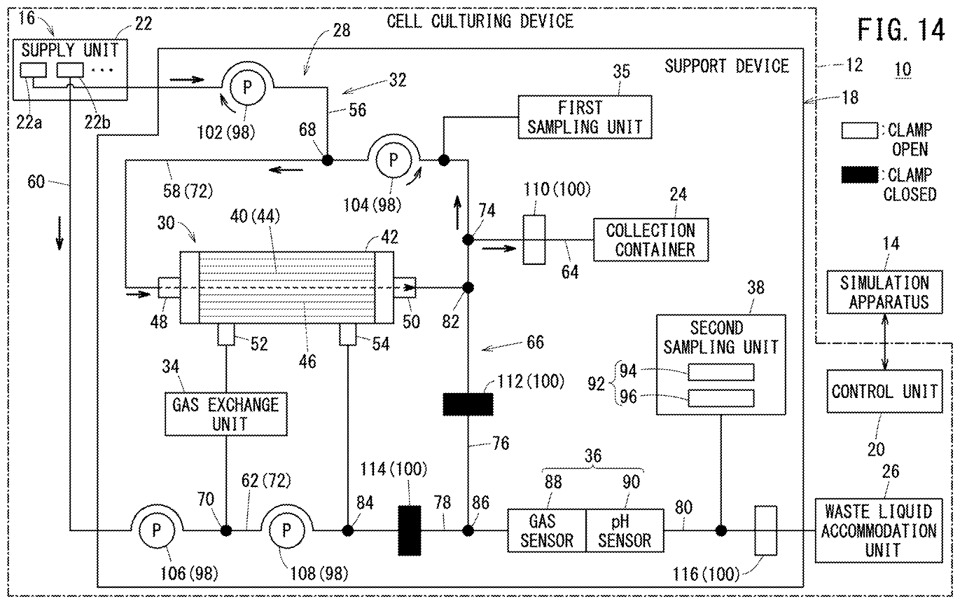

In step S19, the control unit 20 may carry out collection of the cells. As illustrated in FIG. 14, the clamp control unit 124 may control the collection clamp 110 and thereby open the collection flow path 64. Upon doing so, the solution containing the cells inside the first circulation flow path 58 may be guided via the collection flow path 64 into the collection container 24. Consequently, the series of steps of the cell culturing method are completed.

Moreover, as in the determination of YES in step S6 of FIG. 10, it should be noted that steps S1 to S6 may be repeatedly performed. For example, steps S1 to S6 may be performed N times (N is equal to or greater than 2). In the Nth time that step S1 is performed, the results measured in the cell culturing process of step S5 of the (N−1)th time may be used as the propagation data. In this case, the acquisition unit 140 of the simulation unit 132 may acquire the data of the measurement results from the first storage unit 120 of the control unit 20. However, the respective instances of the glucose measurement result and the lactic acid measurement result stored in the first storage unit 120 may include concentration data. Further, the respective instances of the oxygen measurement result and the carbon dioxide measurement result stored in the first storage unit 120 may include partial pressure data. In the case that the input unit 130 has designated the (N−1)th instance of the measurement results, the acquisition unit 140 may convert the concentration data and the partial pressure data into metabolic rate data. The simulation execution unit 142 may simulate the culturing of the cells using the converted data.

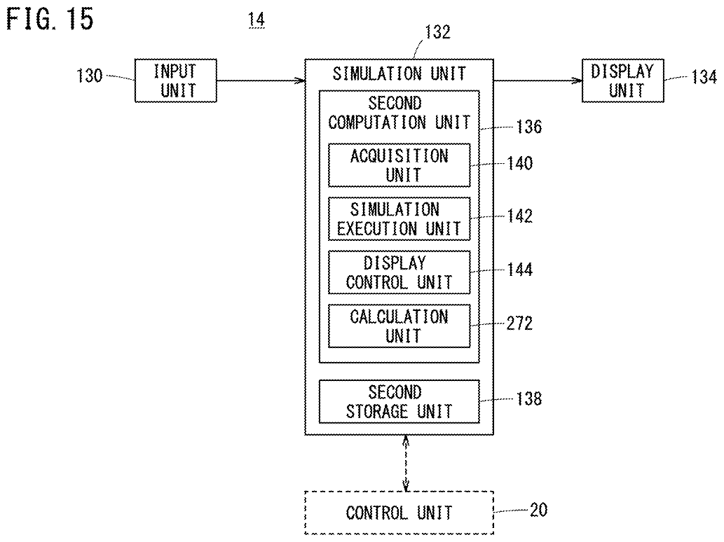

FIG. 15 is a diagram illustrating the configuration of another embodiment of the simulation apparatus 14. As noted previously, the consumption speed of the proteins may be calculated prior to carrying out the cell culturing simulation. The simulation apparatus 14 illustrated in FIG. 15 may include a function of calculating the consumption speed of the proteins.

The second computation unit 136 may also function as a calculation unit 272. The calculation unit 272 may calculate the consumption speed based on the concentrations of the proteins input by the input unit 130. Such a calculation formula may be stored beforehand in the second storage unit 138.

In this case, the user may input the depletion speed (the degradation speed, the elution speed) of each of the proteins in the protein parameter field 182.

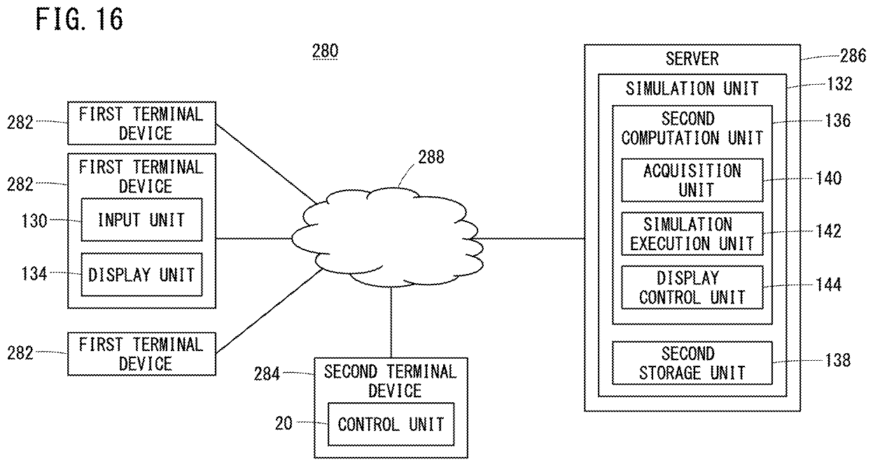

FIG. 16 is a diagram illustrating the configuration of a simulation system 280. The simulation system 280 shown in FIG. 16 may be used instead of the simulation apparatus 14 shown in FIG. 3. In FIG. 16, the same constituent elements as those shown in FIG. 3 are designated by the same reference numerals. The simulation system 280 may include at least one first terminal device 282, at least one second terminal device 284, and a server 286.

A personal computer, a smart phone, a tablet, or the like may be used as the first terminal device 282. The first terminal device 282 may include the input unit 130 and the display unit 134. Further, the first terminal device 282 may also include a processing circuit and a memory, neither of which are shown. The first terminal device 282 may be connected to a communication network 288 via a non-illustrated communication device.

A personal computer, a smart phone, a tablet, or the like may be used as the second terminal device 284. The second terminal device 284 may include the control unit 20. The second terminal device 284 may be connected to the communication network 288 via a non-illustrated communication device.

The server 286 may include the simulation unit 132. The server 286 may be connected to the communication network 288 via a non-illustrated communication device. Moreover, the server 286 may be a cloud server.

The communication network 288 may include a local area network (LAN) or a wide area network (WAN). The first terminal device 282, the second terminal device 284, and the server 286 may be capable of communicating with each other via the communication network 288.

When the user operates the input unit 130 to input data, the first terminal device 282 may transmit each of such data to the server 286. The server 286 may perform the simulation using the data acquired from the first terminal device 282. The server 286 may transmit the results of the simulation to the first terminal device 282. The first terminal device 282 may acquire the results of the simulation from the server 286. The display unit 134 may display the results of the simulation. The second terminal device 284 can acquire data from the server 286.

Moreover, in the simulation system 280, the second computation unit 136 can also function as the calculation unit 272 shown in FIG. 15.

In addition, although it depends on the size of the pores of the hollow fiber membranes 40, it may be difficult for proteins having a large molecular weight (e.g., fibrinogen, fibronectin, and the like) to be eluted from the first region 44 (the culturing region) into the second region 46 (the non-culturing region) and it may be difficult for such proteins to undergo degradation. Concerning such proteins, in the case that the simulation is carried out, at least one of the degradation speed and the elution speed need not necessarily be used.

In the embodiments described above, the present disclosure may be used in order to carry out cell culturing in which the cell culturing device 12 having the hollow fiber membranes 40 is used. The present disclosure can also be used in order to perform cell culturing in which the hollow fiber membranes 40 are not used. For example, the present disclosure can be used to perform shaking culturing, stirring culturing, or the like.

1. A simulation apparatus configured to simulate propagation of cells in a cell culturing device, the simulation apparatus comprising:
an acquisition unit configured to acquire at least one of a depletion speed at which a protein within a culture medium becomes depleted independently of the cells, a consumption speed at which the cells consume the protein under a first culturing condition, and condition data indicating a second culturing condition that differs from the first culturing condition; and
a simulator configured to simulate a change in a concentration of the protein accompanying the propagation of the cells in response to the at least one of the depletion speed, the consumption speed, and the condition data acquired by the acquisition unit.

2. The simulation apparatus of claim 1, wherein
the acquisition unit acquires the depletion speed, the consumption speed, and the condition data, and
the simulator uses the depletion speed, the consumption speed, and the condition data acquired by the acquisition unit to simulate the change in the concentration of the protein accompanying the propagation of the cells.

3. The simulation apparatus of claim 1, wherein the simulation apparatus further includes:
a display configured to display whether the concentration of the protein lies within a predetermined range.

4. The simulation apparatus of claim 3, wherein the simulation apparatus further includes:
a terminal device including the display, and
a server including the acquisition unit and the simulation execution unit,
the terminal device and the server being configured to communicate with each other via a communication network.

5. The simulation apparatus of claim 1, wherein the cell culturing device includes:
a bioreactor;
a hollow fiber membrane disposed in an interior of the bioreactor, the hollow fiber membrane having inner holes;
a culturing region positioned in the inner holes of the hollow fiber membrane;
a non-culturing region positioned in the interior of the bioreactor and externally of the hollow fiber membranes;
a first supply unit configured to supply a culture medium and the cells to the culturing region; and
a second supply unit configured to supply a basal medium free of the protein to the non-culturing region.

6. The simulation apparatus of claim 5, wherein the depletion speed includes:
a degradation speed at which the protein degrades, and
an elution speed at which the protein is eluted from the culturing region into the non-culturing region.

7. The simulation apparatus of claim 1, wherein the simulation apparatus further includes:
an input field configured to receive the depletion speed and the consumption speed.

8. The simulation apparatus of claim 7, wherein the input field is configured to receive the depletion speed and the consumption speed from a user of the cell culturing device.

9. The simulation apparatus of claim 1, wherein the concentration of the protein simulated by the simulator is a first concentration of the protein, and the simulation apparatus further includes:
an input field configured to receive the depletion speed and a change in a second concentration of the protein measured during the first culturing condition; and
a calculation unit configured to calculate the consumption speed based on the second concentration of the protein received by the input field.

10. A simulation system configured to simulate propagation of cells in a cell culturing device, the simulation system comprising:
an acquisition unit configured to acquire a depletion speed at which a protein within a culture medium becomes depleted independently of the cells, a consumption speed at which the cells consume the protein under a first culturing condition, and condition data indicating a second culturing condition that differs from the first culturing condition;
a simulator configured to simulate a change in a concentration of the protein accompanying propagation of the cells during the second culturing condition in response to the depletion speed, the consumption speed, and the condition data acquired by the acquisition unit; and
a display configured to acquire, as a result of the simulation, the concentration of the protein under the second culturing condition and to display whether the concentration of the protein lies within a predetermined range.

11. The simulation system of claim 10, wherein the simulation system further includes:
a terminal device including the display, and
a server including the acquisition unit and the simulation execution unit,
the terminal device and the server being configured to communicate with each other via a communication network.

12. The simulation system of claim 10, wherein the simulation system further includes:
an input field configured to receive the depletion speed and the consumption speed.

13. The simulation system of claim 10, wherein the concentration of the protein simulated by the simulator is a first concentration of the protein, and the simulation apparatus further includes:
an input field configured to receive the depletion speed and a change in a second concentration of the protein measured during the first culturing condition; and
a calculation unit configured to calculate the consumption speed based on the second concentration of the protein input by the input field.

14. The simulation system of claim 10, wherein the cell culturing device includes:
a bioreactor;
a hollow fiber membrane disposed in an interior of the bioreactor, the hollow fiber membrane having inner holes;
a culturing region positioned in the inner holes of the hollow fiber membrane;
a non-culturing region positioned in the interior of the bioreactor and externally of the hollow fiber membranes;
a first supply unit configured to supply a culture medium and the cells to the culturing region; and
a second supply unit configured to supply a basal medium free of the protein to the non-culturing region.

15. A simulation method of simulating propagation of cells in a cell culturing device, the simulation method comprising:
acquiring at least one of a depletion speed at which a protein within a culture medium becomes depleted independently of the cells, a consumption speed at which the cells consume the protein under a first culturing condition, and condition data indicating a second culturing condition that differs from the first culturing condition; and
simulating a change in a concentration of the protein accompanying the propagation of the cells in response to the at least one of the acquired the depletion speed, the consumption speed, and the condition data.

16. The simulation method of claim 15, wherein the simulation method further includes:
displaying whether the concentration of the protein lies within a predetermined range.

17. The simulation method of claim 15, wherein the method includes:
acquiring the depletion speed, the consumption speed, and the condition data; and
simulating a change in the concentration of the protein accompanying the propagation of the cells in response to the depletion speed, the consumption speed, and the condition data.

18. The simulation method of claim 15, wherein the depletion speed includes:
a degradation speed at which the protein degrades, and
an elution speed at which the protein is eluted from the culturing region into the non-culturing region.

19. The simulation method of claim 15, wherein the concentration of the protein is a first concentration of the protein, and the method further includes:
measuring a second concentration of the protein during the first culturing condition; and
calculating the consumption speed based on the second concentration of the protein.

20. The simulation method of claim 15, wherein the at least one of the acquired the depletion speed, the consumption speed, and the condition data is acquired from a user input.
